(19) United States
(12) Patent Application Publication
(10) Pub. No.: US 2016/0183873 A1
(43) Pub. Date: Jun. 30, 2016

(54) FETAL HEALTH MONITORING BELT

(71) Applicant: HON HAI PRECISION INDUSTRY CO., LTD., New Taipei (TW)

(72) Inventors: YI-CHENG LIN, New Taipei (TW); TUAN-CHUN CHEN, New Taipei (TW); CHANG-DA HO, New Taipei (TW); HSIN-PEI HSIEH, New Taipei (TW); JEN-TSORNG CHANG, New Taipei (TW)

(21) Appl. No.: 14/692,167

(22) Filed: Apr. 21, 2015

(30) Foreign Application Priority Data
Dec. 31, 2014 (TW) 103146680

(51) Int. Cl. A61B 5/00; A61B 5/0408; A61B 5/0205; A61B 5/04; A61B 5/11; A61B 8/08; A61B 8/00

(52) U.S. Cl. CPC A61B 5/6831; A61B 8/0883; A61B 8/0866; A61B 5/04085; A61B 8/54; A61B 5/04012; A61B 5/4356; A61B 5/11; A61B 5/0205; A61B 5/7246; A61B 5/6823; A61B 2560/0475

(57) ABSTRACT

A fetal health monitoring belt includes a main body and a fixing part, wherein the main body includes an ultrasonic sensor, at least one ECG sensor and a microcontroller which includes a control module, a signal processing module and a data transmitting module, and the control module electrical connects the ultrasonic sensor. The ultrasonic sensor detects the location of the beating heart of the fetus, and transmits a location signal to the control module. The control module receives the location signal and transmits a detection signal to the corresponding ECG sensor. The ECG sensor detects the ECG signal and processes the signal by the signal processing module, and transmits the signal by the data transmitting module.

### FIELD

The subject matter herein generally relates to a fetal health monitoring systems, particularly to a fetal health monitoring belt capable of detecting the electrocardiogram (ECG) of fetus.

### BACKGROUND

Fetal electrocardiogram (ECG) is an important tool for detecting the health condition of a fetus. Because the fetus continuously moves in a mother's womb, the location of the fetal heart moves as well.

### BRIEF DESCRIPTION OF THE DRAWINGS

Implementations of the present technology will now be described, by way of example only, with reference to the attached figures, wherein:

FIG. 1 is an isometric view of the fetal health monitoring belt of present disclosure.

FIG. 2 is a block diagram of the fetal health monitoring belt of FIG. 1.

FIG. 3 is a block diagram of the signal processing module of the fetal health monitoring belt of FIG. 2.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures and components have not been described in detail so as not to obscure the related relevant feature being described. Also, the description is not to be considered as limiting the scope of the embodiments described herein. The drawings are not necessarily to scale and the proportions of certain parts have been exaggerated to better illustrate details and features of the present disclosure.

Several definitions that apply throughout this disclosure will now be presented.

The term “comprising,” when utilized, means “including, but not necessarily limited to”; it specifically indicates open-ended inclusion or membership in the so-described combination, group, series and the like.

In general, the word “module”, as used herein, refers to logic embodied in hardware or firmware, or to a collection of software instructions, written in a programming language, such as, Java, C, or assembly. One or more software instructions in the modules may be embedded in firmware, such as in an EPROM. The modules described herein may be implemented as either software and/or hardware modules and may be stored in any type of non-transitory computer-readable medium or other storage system. Some non-limiting examples of non-transitory computer-readable medium include CDs, DVDs, BLU-RAY, flash memory, and hard disk drives.

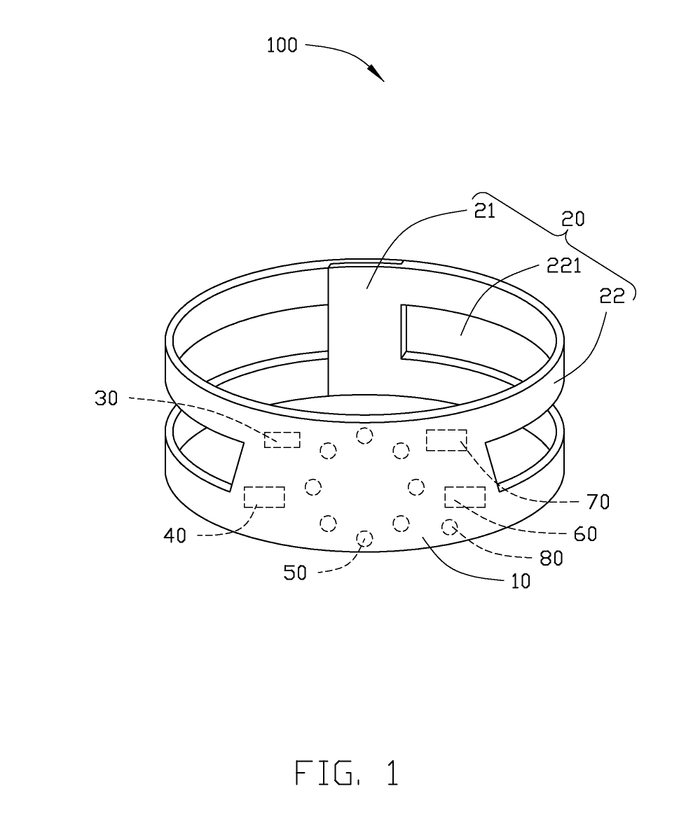

FIG. 1 illustrates an isometric view of the fetal health monitoring belt 100 of present disclosure. The fetal health monitoring belt 100 comprises a main body 10 and a fixing part 20. The fixing part 20 comprises two fastening parts 21 and two connecting parts 22. A strip opening 221 is defined on each connecting part 22 to lighten the weight of the fetal health monitoring belt 100.

The main body 10 connects with two fastening parts 21 and two connecting parts 22 to support the abdomen of a pregnant woman and protect the fetus.

In this embodiment, the main body 10 of the fetal health monitoring belt 100 further comprises a motion sensor 30, an ultrasonic sensor 40, a plurality of ECG sensors 50, a microcontroller 60, a power supply module 70 and a switch 80.

In other embodiments, an ultrasonic emitter and an ultrasonic receiver can replace the ultrasonic sensor 40 of the embodiment of FIG. 1.

FIG. 1 illustrates the motion sensor 30 is electrically connected with the microcontroller 60. The motion sensor 30 is used to detect the motion signal of the fetus, and transmit the detected signal to the microcontroller 60.

The ultrasonic sensor 40 is electrically connected to the microcontroller 60. The ultrasonic sensor 40 is used to transmit an ultrasound and receive the reflected ultrasonic signal from the fetus, and transmit the position of the heart and the figure signal of the fetus to the microcontroller 60. The plurality of the ECG sensors 50 which electrically connect with the microcontroller 510 are used to detect the ECG signals of the fetus. In this embodiment, the number of the ECG sensors 50 is eight, and configured in a ring shape at the middle part of the main body 10.

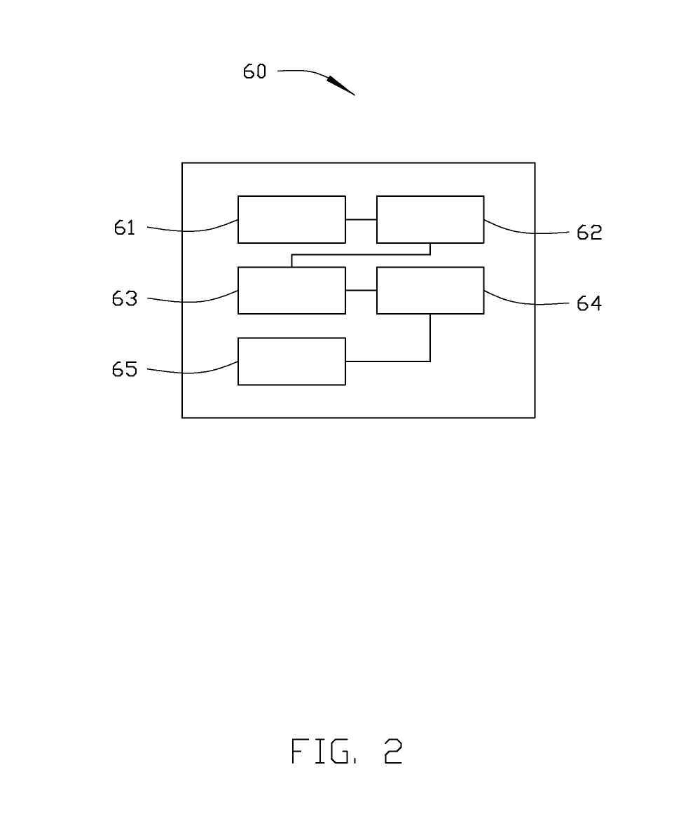

FIG. 2 illustrates a block diagram of the fetal health monitoring belt of FIG. 1. The microcontroller 60 comprises a control module 61, a signal processing module 62, a data storage module 63, a data comparison module 64 and a data transmitting module 65.

The control module 61 which electrical connects with the ultrasonic sensor 40 is used to receive the heart location signal of the fetus in the mother's womb which is detected by the ultrasonic sensor 40, and is used to control the corresponding ECG sensor 50 to execute a ECG test. In this embodiment, the control module 61 controls the ECG sensor 61 which has the least distance with the location of the heart to detect the ECG signal.

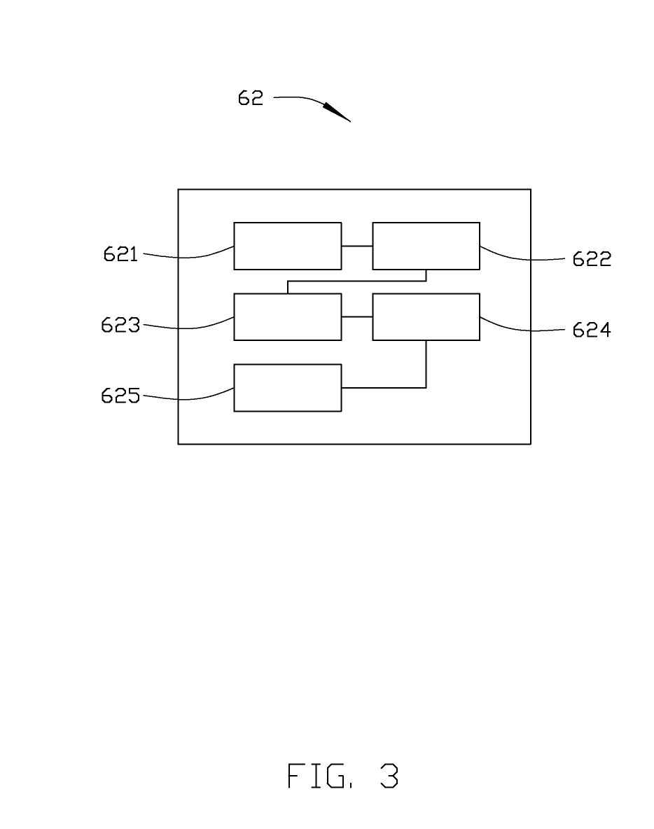

FIG. 3 illustrates a block diagram of the signal processing module of the fetal health monitoring belt of FIG. 2. The signal processing module 62 comprises a signal receiving unit 621, a signal amplifying unit 622, an analog digital converter (ADC) unit 623, a signal separating unit 624 and a signal analyzing unit 625.

The signal receiving unit 621 is used to receive the action signal transmitted from the motion sensor 30, the figure signal of the fetus transmitted from the ultrasonic sensor 40 and the ECG signal transmitted from the ECG sensor 50.

The signal amplifying unit 622 is used to amplify the signal received from the signal receiving unit 621 to be recognized by the ADC unit 623.

The ADC unit 623 is used to convert the amplified signal to a number signal suitable for the signal separating unit 624.

The signal separating unit 624 is used to separate the signals from mother and the fetus.

The signal analyzing unit 625 is used to analyze the signal separated from the signal separating unit 624, and further analyzes the figure of the fetus, the motion rhythm, heart beating and the uterine contraction of mother.

The data storage module 63 is used to store the data obtained from the signal analyzing unit 625, and the data storage module 63 is preprogrammed with fetal health data criteria of different development stages. In one embodiment, the data storage module 63 can be an internal storage device, such as a random access memory (RAM) for temporary storage of information, and/or a read only memory (ROM) for permanent storage of information. In some embodiments, the storage module 63 may also be an external storage device, such as an external hard disk, a storage card, or a data storage medium.

The data comparison module 64 is used to compare the obtained data of the fetus with the health data criteria of different stages which is preprogrammed, and gives the comparison results.

The data transmitting module 65 is used to transmit the obtained data and the compare result to the cloud. In this embodiment, the data transmitting module 65 is BLUETOOTH™.

Referring also to FIG. 1, the power supply module 70 is used to supply the power to the components of the fetal health monitoring belt 100.

The switch 80 which electrically connects with the power supply module 70 is used to control the power supply module 70 supplying the power to the components of the fetal health monitoring belt 100. For example, the user pushes the switch 80, the power supply module 70 starts to supply power to the components of the fetal health monitoring belt 100, therefore the components of the fetal health monitoring belt 100 start to execute the function.

For example, the ultrasonic sensor 40 transmits ultrasound and receives a reflected ultrasound from the fetus, and further detects the location signal of the heart of the fetus and the figure signal of the fetus. The ultrasonic sensor 40 transmits the location signal of the heart to the control module 61, and transmits the figure signal of the fetus to the signal receiving unit 621. The control module 61 transmits a detecting signal to the ECG sensor 50 which has the least distance with the location of the heart of the fetus to detect an ECG signal, and also transmits the detected ECG signal to the signal receiving unit 621.

Meanwhile, the motion sensor 30 starts to detect the motion signal of the fetus, and transmit the motion signal to the signal receiving unit 621.

The signal received by the signal receiving unit 621 is amplified by the signal amplifying unit 622; converted by the ADC unit 623; separated by the signal separating unit 624; analyzed by the analyzing module 625, and then obtains data including the motion signal of the fetus, heart beat data, ECG and the uterine contraction of mother, and then store the data in the storage module 63. The data comparison modules 64 compares the obtained data with the preprogrammed health data criteria and obtains a result, the result is transmitted to the cloud by the data transmitting module 65. A doctor and a user can access the data by using a mobile device, for example, a cell phone.

The fetal health monitoring belt of present disclosure first detects the location of the heart and then detects the ECG signal of the fetus; therefore the ECG signal has increased accuracy.

The embodiments shown and described above are only examples. Many details are often found in the art such as the other features of a fetal health monitoring belt. Therefore, many such details are neither shown nor described. Even though numerous characteristics and advantages of the present technology have been set forth in the foregoing description, together with details of the structure and function of the present disclosure, the disclosure is illustrative only, and changes may be made in the detail, especially in matters of shape, size and arrangement of the parts within the principles of the present disclosure up to, and including_{—} the full extent established by the broad general meaning of the terms used in the claims. It will therefore be appreciated that the embodiments described above may be modified within the scope of the claims.

What is claimed is:

1. A fetal health monitoring belt comprising:
a main body and a fixing part configured to fasten the fetal health monitoring belt on the maternal abdomen, the main body comprising:
an ultrasonic sensor, at least one ECG sensor and a microcontroller which comprises a control module, a signal processing module and a data transmitting module, wherein the control module electrical is connected to the ultrasonic sensor,
wherein the ultrasonic sensor is configured to detect a location from which a heartbeat of a fetus emanates and transmit a location signal to the control module, the control module is configured to receive the location signal and transmit a detection signal to the ECG sensor, and the ECG sensor is configured to detect a ECG signal, the signal processing module is configured to process the detected ECG signal and the data transmitting module is configured to transmit the detected ECG signal to a receiving end.

2. The fetal health monitoring belt of claim 1, wherein the ultrasonic sensor further detecting the figure of the fetus and transfers the figure information to the signal processing module as a figure signal.

3. The fetal health monitoring system of claim 2, wherein the signal processing module comprising: a signal receiving unit, a signal amplifying unit, a analog digital converter unit, a signal separating unit and a signal analyzing unit.

4. The fetal health monitoring belt of claim 3, wherein the signal receiving unit is configured to receive the ECG signal and the figure signal; the signal amplifying unit is configured to amplify the signal which is received from the signal receiving unit to a proper multiple to recognize by the analog digital converter unit; the analog digital converter unit is configured to transfer the amplified signal to a number signal which is suitable for being recognized by the signal separating unit; the signal separating unit is configured to separate a signal from the mother and a signal from the fetus, the signal analyzing unit is configured to analyze the signal which is separated by the signal separating unit and further analyzing the figure of the fetus, the hearting, the ECG and the situation of uterine contraction.

5. The fetal health monitoring belt of claim 3, wherein the microcontroller further comprising a motion sensor is configured to sense the motion signal of the fetus; the motion signal is received by the signal receiving unit and then is amplified by the signal amplified unit to a multiple which is suitable for the analog digital converter unit to transfer it to a number signal, and then the signal separating unit the number signal, and further analyzed by the signal analyzing unit to obtain a motion information of the fetus.

6. The fetal health monitoring belt of claim 2, wherein the microcontroller further comprises a data storage module which is configured to storage the data obtained from the signal analyzing unit, and the data storage module is preprogrammed with fetal health data criteria at different stage.

7. The fetal health monitoring belt of claim 2, wherein the microcontroller further comprising a data comparison module which is configured to compare the obtained data with the preprogrammed fetal health data criteria, and shows the result.

8. The fetal health monitoring belt of claim 1, wherein the ECG sensors are configured in the middle portion of the main body in a ring shape.

9. The fetal health monitoring belt of claim 1, wherein the fetal health monitoring belt further comprising a power supply module and a switch which connects with the power supply module.

10. The fetal health monitoring belt of claim 1, wherein the fixing part comprising two fastening parts and two connecting parts, the connecting parts separately are connecting to the two ends of the main body and two fastening parts, a strip of opening is defined on every connecting part to lighten the weight of the belt.
